(19) United States
(12) Patent Application Publication
(10) Pub. No.: US 2012/0167882 A1
(43) Pub. Date: Jul. 5, 2012

(54) TEMPERATURE MONITORING AND CONTROL DEVICES FOR TRACHEAL TUBES

(75) Inventors: Lockett E. Wood, Lyons, CO (US); Sarah Hayman, Boulder, CO (US)

(73) Assignee: Nellcor Puritan Bennett LLC, Boulder, CO (US)

(21) Appl. No.: 12/980,665

(22) Filed: Dec. 29, 2010

(51) Int. Cl. A61M 16/04

(52) U.S. Cl. 128/204.17

(57) ABSTRACT

Various embodiments of an intubation system include a tracheal tube, a heat source coupled to the tracheal tube, and a temperature sensor disposable in a patient's trachea to detect a temperature within the patient's trachea. The heat source is adapted to generate heat when the tracheal tube is disposed in the airway of the patient. A temperature control system coupled to the heat source is adapted to monitor the detected temperature and to control generation of heat from the heat source based on the detected temperature.

### BACKGROUND

The present disclosure relates generally to medical devices and, more particularly, to temperature monitoring and control devices for tracheal tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into and out of the patient. For example, medical devices, such as tracheal tubes, may be used to control the flow of air or other gases through a trachea of a patient. Such tracheal tubes may include endrotracheal tubes (ETTs), or tracheostomy tubes. In many instances, it is desirable to provide a seal between the outside of the tube or device and the interior of the passage in which the tube or device is inserted, such as the trachea. In this way, substances can only flow through the passage via the tube or other medical device inserted in the tube, allowing a medical practitioner to maintain control over the type and amount of substances flowing into and out of the patient.

Depending on the clinical application, some tracheal tubes may be equipped with devices, such as cameras, fiber-optics, light sources, transducers, and so forth, which generate heat during operation. While such devices may serve a clinical need, for example, by aiding in visualization of the patient's anatomy during tracheal tube placement, the heat dissipated by such devices may rise above desired temperatures within the patient. In some instances, the mechanical structure in which the heat generating device is provided may distribute or dissipate the generated heat away from the body tissue.

### BRIEF DESCRIPTION OF THE DRAWINGS

Advantages of the disclosed techniques may become apparent upon reading the following detailed description and upon reference to the drawings in which:

FIG. 1 is an elevational view of an endobronchial tube including a heat generating device, a temperature sensor, and a monitoring and control system;

FIG. 2 is a is a method that may be utilized to operate the double lumen endobronchial tube of FIG. 1;

FIG. 3 is a flow chart illustrating a method that may be utilized by the control circuitry of FIG. 1 to control operation of the one or more electronic devices coupled to the endobronchial tube; and

FIG. 4 illustrates a method that may be utilized by the control circuitry of FIG. 1 to substantially reduce or prevent overheating.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, embodiments of a tracheal tube including a temperature sensing device, a heat generating device, and a temperature control and monitoring system are provided herein. In one embodiment, the tracheal tube may be an endobronchial tube, and the electronic heat generating devices may be a camera and an illumination device coupled to the endobronchial tube via a collar. Endobronchial tubes are double-lumen tracheal tubes that facilitate an airtight seal in the trachea and one stem of a patient bronchus to allow independent ventilation of one lung. Generally, an endobronchial tube includes two tubes of unequal length that are attached. One tube terminates within the tracheal airway space, i.e., the shorter tube has a distal end at a location similar to a typical endotracheal tube. The other, longer, tube is configured to extend past the shorter tube and into a left or right bronchial stem. Both tubes define a passageway for transferring gases to and from a patient, and the endobronchial tube must be positioned correctly relative to the anatomy for proper functioning. In some embodiments, during placement of such devices, the camera and the illumination device may be utilized to assist the operator in the proper placement of the endobronchial tube by facilitating visualization of the patient's anatomy. The temperature sensor may be configured to sense a temperature indicative of a temperature of the patient's tissue while the heat generating devices are being utilized. The control and monitoring system monitors the sensed temperature and, if necessary, alters one or more parameters of the heat generating devices to maintain the sensed temperature in a desired range.

The foregoing features of embodiments of the disclosed systems and methods may be advantageous in medical applications in which one or more heat generating devices are utilized within a patient and are placed in contact with a patient's tissue (e.g., the tracheal mucosa). For instance, as understood by one skilled in the art, the extent of exposure of a heat generating device composed of a given material to a patient's tissue may be a function of temperature. For example, as set forth by the International Organization for Standardization (ISO) in standard number 60601-1, a heat generating device made of metal, liquid, glass, porcelain, vitreous material, moulded material, plastic, rubber, or wood that is in contact with human skin for greater than 10 minutes should not exceed a temperature of 43° C. For further example, as set forth by ISO 60601-1, a heat generating device made of the aforementioned materials that is in contact with human skin for a time interval less than 10 minutes but greater than or equal to 1 minute should not exceed a temperature of 48° C. As such, embodiments of the present invention may be adapted to monitor the length of time a heat generating device is in contact with a portion of a patient's tissue as well as a temperature level indicative of the temperature of the patient's tissue.

The tracheal tubes provided herein may be disposable rather than reusable, capable of conveying gas to and from the patient, and capable of providing separate ventilation channels to the tracheal space and to an individual lung. It should be noted that the provided tracheal tubes and methods of operating the tracheal tubes may be used in conjunction with auxiliary devices, such as airway accessories, ventilators, humidifiers, and so forth, which may cooperate with the tracheal tubes to maintain airflow to and from the lungs of the patient. For instance, the tracheal tubes may be placed in the trachea and coupled to a ventilator to protect the airway from possible obstruction or occlusion in emergency situations, such as when a patient experiences cardiac or respiratory arrest. For further example, the tracheal tubes may be coupled to an adapter or connector that is configured to cooperate with control circuitry to activate valving that controls the airflow to and from the patient during inspiration and expiration.

Furthermore, although the embodiments of the present disclosure illustrated and described herein are discussed in the context of endobronchial tubes, it should be noted that presently contemplated embodiments may include a temperature control and monitoring system coupled to a temperature sensor and one or more heat generating devices associated with any of a variety of suitable devices. For example, the temperature control and monitoring systems and devices described herein may be associated with a tracheostomy tube, a Broncho-Cath™ tube, a specialty tube, a laryngoscope, a supraglottic airway tube, or other airway devices. Indeed, any device with a ventilation lumen designed for use in an airway of a patient may include the temperature control and monitoring devices described herein. Furthermore, as used herein, the term “tracheal tube” may include an endotracheal tube, a tracheostomy tube, an endobronchial tube (e.g., Broncho-Cath™ tube), a specialty tube, or any other airway device.

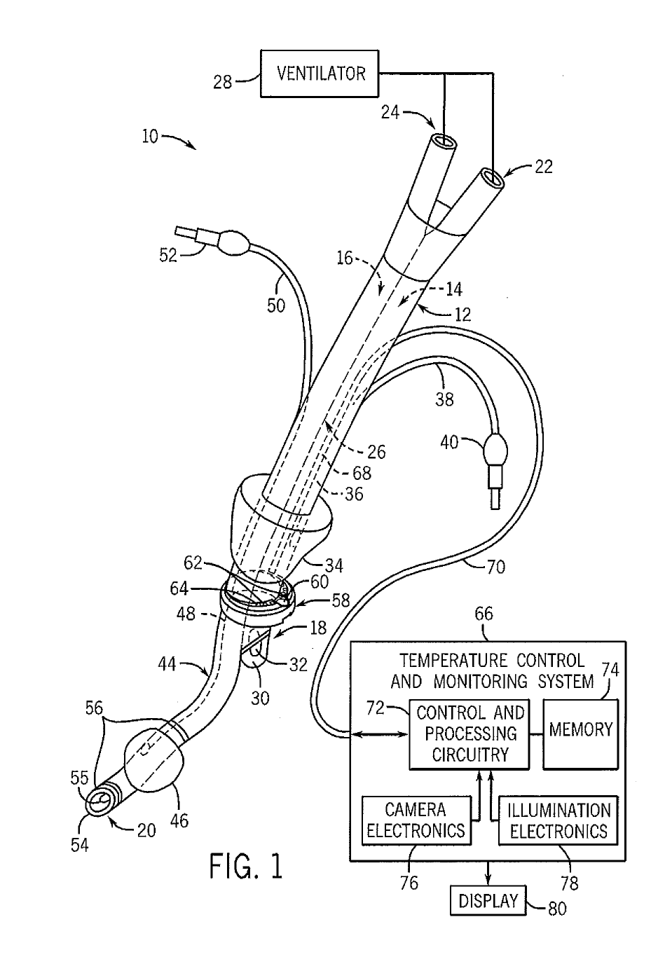

Turning now to the drawings, FIG. 1 is an elevational view of an exemplary tracheal tube 10 configured to be placed in a patient bronchial stern in accordance with aspects of the present disclosure. The tracheal tube 10 includes a central tubular body 12 with a tracheal ventilation lumen 14 and a bronchial ventilation lumen 16. The tracheal lumen terminates at a tracheal lumen distal end 18 while the bronchial lumen terminates in a bronchial lumen distal end 20. Furthermore, the tracheal tube 10 may include a tracheal lumen proximal end 22 and a bronchial lumen proximal end 24. As shown, the tracheal ventilation lumen 14 and a bronchial ventilation lumen 16 may be attached to one another over a portion of the tubular body 12 and may separate at their respective proximal ends 22, 24 and distal ends 18, 20.

The tracheal lumen proximal end 22 and a bronchial lumen proximal end 24 may be outfitted with separate connectors that may be attached to a ventilation device 28 during operation. The ventilation device 28 may include a suitable controller (e.g., a processor-based control system) so that a clinician may direct airflow to and from both the tracheal ventilation lumen 14 and bronchial ventilation lumen 16. In other embodiments, either the tracheal ventilation lumen 14 or the bronchial ventilation lumen 16 may be blocked or otherwise closed such that only one of the two lumens of the tracheal tube 10 is operational.

The tracheal lumen distal end 18 of ventilation lumen 14 terminates in an opening 30 and may be placed in a patient trachea during operation to maintain airflow to and from the patient's lungs. A Murphy's eye 32 may optionally be present and may be located on the ventilation lumen 14 opposite the opening 30 to prevent airway occlusion when the tracheal tube assembly 10 is improperly placed within the patient's trachea. As illustrated, a tracheal cuff 34 may encircle the tubular body 12 and be inflated to seal against the walls of a body cavity (e.g., a trachea). The cuff 34 may be inflated via an inflation lumen 36 terminating in an inflation tube 38 connected to an inflation pilot balloon and valve assembly 40. Additionally, it should be noted that the cuff 34 may be any suitable cuff, such as a tapered cuff, a non-tapered cuff, and so forth. The tracheal ventilation lumen 14 may also include a suction lumen (not shown) that extends from a location on the tracheal tube 10 positioned outside the body when in use to a location on the tubular body 12 that terminates in a port located proximally to cuff 34 through which secretions may be aspirated. Bronchial ventilation lumen 16 is longer than tracheal ventilation lumen 14 and includes a distal portion 44 that extends past the tracheal lumen distal end 18. The bronchial ventilation lumen 16 may include a bronchial inflation cuff 46 that is configured to seal against the walls of a patient's bronchial stem. The cuff 46 may be inflated via an inflation lumen 48 terminating in an inflation tube 50 connected to an inflation pilot balloon and valve assembly 52.

The tubular body 12 and the cuff 34 may be formed from materials having desirable mechanical properties (e.g., puncture resistance, pin hole resistance, tensile strength, and so forth) and desirable chemical properties (e.g., biocompatibility). Further, in one embodiment, the walls of the cuff 34 or the cuff 46 may be made of a polyurethane (e.g., Dow Pellethane® 2363-80A) having suitable mechanical and chemical properties. In other embodiments, the walls of the cuff 34 or the cuff 46 may be made of silicone or a suitable polyvinyl chloride (PVC). In certain embodiments, the cuff 34 or the cuff 46 may be generally sized and shaped as a high volume, low pressure cuff that may be designed to be inflated to pressures between approximately 15 cm H2O and 30 cm H2O. Further, bronchial cuff 46 may be a different color or include other identifying markings that allow a user to differentiate between the tracheal cuff 34 and the bronchial cuff 46. In addition, in some embodiments, to assist in proper placement of the tube 10, x-ray visible markings 56 may be placed at any appropriate location. For example, the markings 56 may outline a bronchial distal opening 54 or a side eye 55.

Still further, in the illustrated embodiment, a collar 58 encircles the tubular body 12 in a location below the cuff 34. As shown, the illustrated collar 58 includes a camera 60 that is provided for visualization of the patient's anatomy as the double lumen tracheal tube 10 is inserted into the patient. The collar 58 also includes illumination devices 62, which provide illumination for the camera 60, and a temperature sensor 64 adapted to sense an environmental temperature. In some embodiments, the temperature sensor 64 may be placed in a location on the collar 58 that is suitable for measurement of a temperature level representative of the temperature of the patient's tissue (e.g., temperature of the tracheal mucosa). To that end, the temperature sensor 64 may be any suitable device capable of measuring temperature when placed within the patient, such as a thermistor, a thermocouple, a semiconductor, and so forth.

It should be noted that although in the illustrated embodiment, the camera 60, the illumination devices 62, and the temperature sensor 64 are disposed on the collar 58, in other embodiments, such devices may be located in any desirable location on the tube 10. Indeed, some or all of the illustrated components may not be present in all embodiments, and such components may not be mounted on a collar. For example, in one embodiment, the collar 58 may exclusively include the illumination devices 62 and the temperature sensor 64. In another embodiment, the collar 58 may exclusively include the camera 60 and the temperature sensor 64. Still further, in additional embodiments, other electronic devices configured to function as a heat source during operation may be mounted on the collar 58 or otherwise associated with the tube 10. Indeed, certain embodiments may include the temperature sensor 64 and any desired electronic heat source device configured for any desirable purpose.

The collar 58 and the components mounted thereon are coupled to a temperature control and monitoring system 66 via a lumen 68 terminating in a tube 70. The temperature control and monitoring system 66 is provided to monitor and control the heat generated by the electronic devices disposed on the collar 58 to substantially reduce or prevent the likelihood of overheating. To that end, the control and monitoring system 66 includes control and processing circuitry 72 associated with memory 74, camera electronics 76, and illumination electronics 78. The control and monitoring system 66 is also associated with a display 80 that is utilized to communicate information regarding operation of the electronic devices disposed on the collar 58.

During operation, the tracheal tube 10 is inserted into the trachea of a patient and positioned within the left or right bronchial stem, and the tracheal cuff 34 and bronchial cuff 46 are inflated to isolate the appropriate airway structures. The camera 60 and the illumination devices 62 are operated to visualize the patient's anatomy, for example, during placement of the tracheal tube 10. In the illustrated embodiment, such devices are controlled by the control and monitoring system 66, which is located outside the patient's body when the patient is intubated, via control wires located in the lumen 68. For example, the camera electronics 76 located in the control system 66 provide control and power for the camera 60, and the illumination electronics 78 provide control and power for the illumination devices 62. For further example, the camera electronics 76 may exhibit control over one or more parameters (e.g., duty cycle) of the camera 60 to control its operation. Likewise, the illumination electronics 78 may control a parameter, such as a duty cycle, of the illumination devices 62 to control their functionality.

Operation of the electronic heat sources (e.g., the camera and the illumination devices) generates heat within the patient when intubated. As such devices are continually operated throughout the intubation period of the patient, a rise in the overall heat level to which the patient's tissue is exposed may occur. The temperature control and monitoring system 66 may be configured to monitor a temperature within the patient and to control operation of the electronic devices disposed therein to substantially maintain the temperature of the patient's tissue within a predetermined acceptable temperature range. To that end, the temperature sensor 64 operates concurrently with the heat generating devices (e.g., the camera and the illumination devices) to detect a temperature indicative of the temperature of the patient's tissue and to communicate the detected temperature to the control and processing circuitry 72. The control and processing circuitry 72 is configured to monitor the detected temperature over time, to compare the monitored temperature to predetermined threshold values, and to alter one or more parameters of the heat generating devices to maintain the detected temperature within a desired range, as described in more detail below.

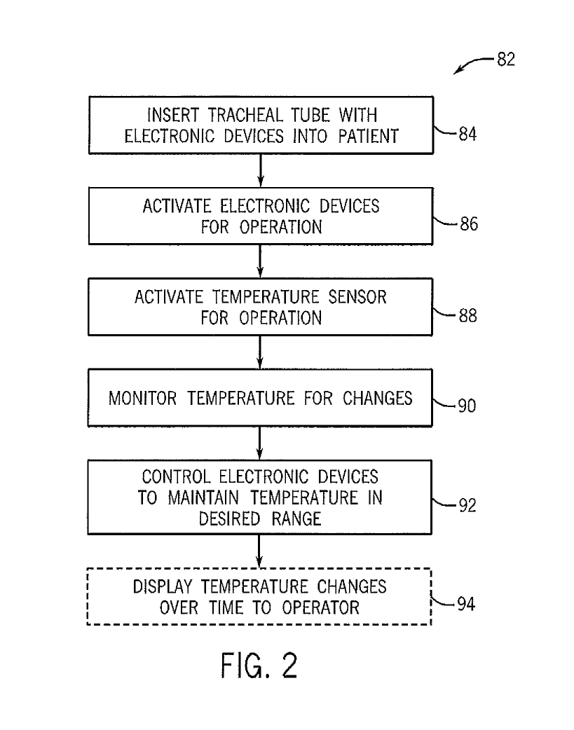

FIG. 2 is a method 82 that may be utilized to operate the tracheal tube 10 of FIG. 1. The method 82 includes inserting the tracheal tube 10 with the mounted electronic devices into a patient (block 84) and activating the electronic devices for operation (block 86). For example, during use of the tracheal tube 10 of FIG. 1, the camera 60 and the illumination devices 62 are powered ON for use. The method 82 further includes activating the temperature sensor for operation (block 88) and monitoring the detected temperature for changes (block 90). For example, as described in more detail below, the temperature may be monitored to determine whether or not one or more predefined temperature thresholds have been exceeded. Again, the temperature sensor is typically placed in a location representative of the temperature of a patient's tissue, and, accordingly, by monitoring the detected temperature, the control system may monitor an indicator of the temperature of a patient's tissue. For example, in some embodiments, the temperature sensor may be placed in a location in contact with the patient's tissue. In other embodiments, the temperature sensor may be placed in a location proximate to but not in direct contact with the patient's tissue, and the detected temperature may be utilized to derive or estimate the temperature of the patient's tissue.

Additionally, the method 82 includes controlling the electronic devices disposed in the patient's trachea to maintain the detected temperature, a calculated temperature change, and/or a rate of change of temperature over time within a desired range (block 92). For example, in one embodiment, a temperature change may be limited to approximately 4 degrees Celsius, and if the detected temperature changes by more than 4 degrees within a predefined number of samplings, the controller may implement control to attempt to reduce the temperature. Still further, the method 82 also includes displaying the temperature changes over time to an operator (block 94) if desired. It should be noted that additional information, such as length of time the electronic devices have been active, changes in parameters of the electronic devices, and so forth, may also be displayed to the operator if desired.

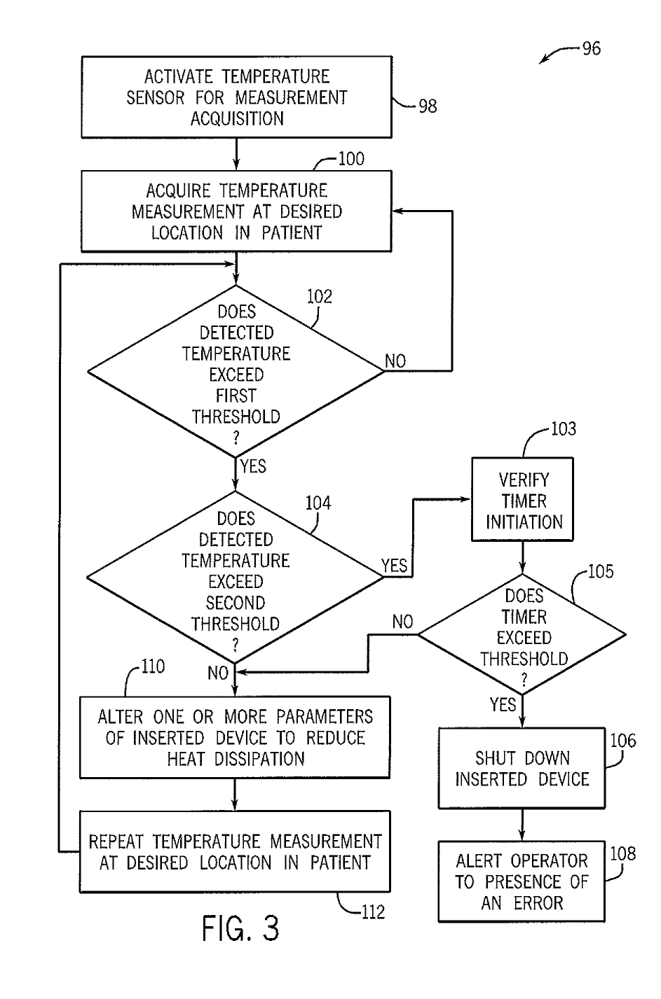

FIG. 3 is a flow chart illustrating an exemplary method 96 that may be utilized by the control circuitry 72 of FIG. 1 to control operation of the one or more electronic devices coupled to the tracheal tube 10. The method 96 includes activating the temperature sensor for measurement acquisition (block 98) and acquiring a temperature measurement at the desired location in the patient (block 100). The control circuitry then checks if the detected temperature exceeds a first threshold (block 102), for example, a first temperature threshold equal to approximately 39° C. If the temperature is below the predefined threshold, the control circuitry continues to acquire temperature measurements and check whether or not such measurements exceed the given threshold. However, in the illustrated embodiment, if the temperature does exceed the first threshold, the control circuitry checks whether the detected temperature exceeds a desired threshold (block 104). It should be noted that in other embodiments, any number of threshold values may be utilized by the control circuitry.

In the illustrated embodiment, if the detected temperature exceeds the second threshold, the control circuitry verifies that a timer has been initiated (block 103) and checks whether the timer exceeds a predefined threshold (block 105). In some embodiments, the foregoing step may enable the control circuitry to adjust one or more parameters of the inserted device to reduce heat dissipation (block 110) instead of shutting down the device when the detected temperature exceeds the second threshold. However, if the timer does exceed the predefined threshold (e.g., 60 seconds), the inserted electronic devices (e.g., a camera or illumination device) are shut down (block 106) and prevented from being operated to produce additional heat. Such a step may reduce or prevent the likelihood of overheating in instances in which the temperature rises above a desired threshold, such as approximately 40° C. Subsequently, the operator is alerted to the presence of an elevated temperature (block 108).

If the detected temperature exceeds the first threshold but does not exceed the desired threshold, the control circuitry alters one or more parameters of the inserted electronic device to reduce heat dissipation (block 110). For example, in one embodiment, the control circuitry may reduce the amount of time an inserted camera and/or illumination device is powered ON, thus altering the duty cycle of one or both of the devices and reducing the amount of heat generated by such devices. After altering a parameter of the inserted device, the control circuitry repeats the temperature measurement at the desired location in the patient (block 112) and again checks whether or not the detected temperature exceeds one or both of the predefined thresholds. In such a way, the control circuitry may be configured to continuously monitor the detected temperature during intubation, to alter parameters to maintain the detected temperature within a desired range, and to deactivate device operation to reduce the likelihood of overheating when a desired threshold is exceeded.

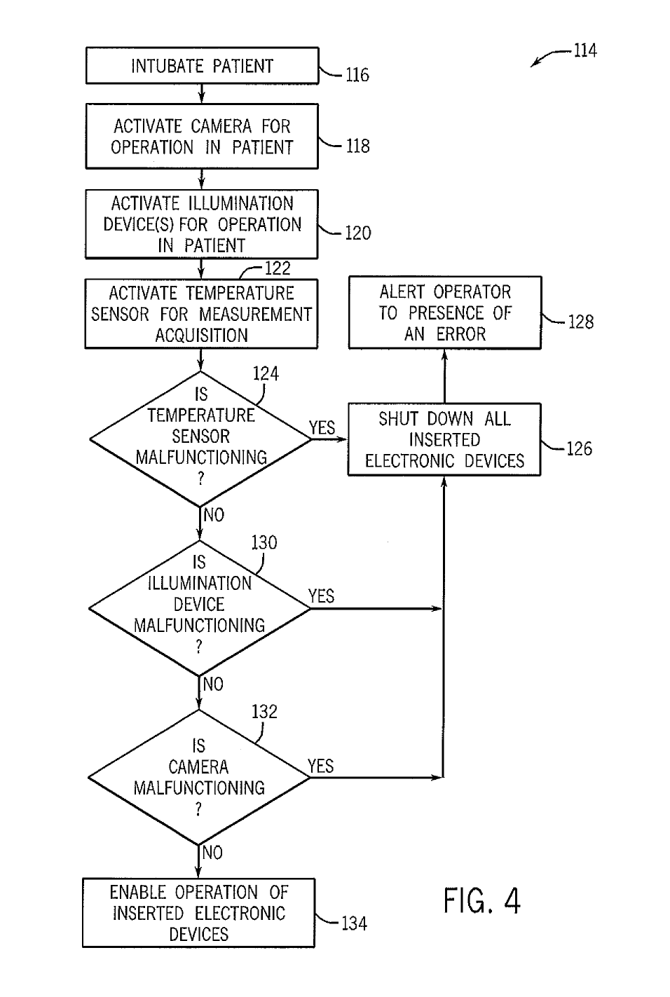

FIG. 4 illustrates a method 114 that may be utilized by the control circuitry of FIG. 1 to substantially reduce or prevent the likelihood of resulting from operation or malfunction of one or more of the inserted electronic devices. It should be noted that the illustrated method relates to operation of the embodiment of FIG. 1 after insertion into the patient. However, such a method may be utilized with any airway device having one or more heat generating devices and a temperature sensor. The illustrated method 114 includes intubating the patient (block 116) and activating the camera for operation in the patient (block 118). The method 114 further includes activating the illumination devices for operation (block 120) and activating the temperature sensor for measurement acquisition (block 122). In some embodiments, such steps may be performed before or during the intubation process such that the camera and illumination devices may be utilized to guide insertion of the tracheal tube.

The method 114 also includes checking if the temperature sensor is malfunctioning (block 124) and, if so, shutting down all inserted electronic devices (block 126) and alerting the operator to the presence of an error (block 128). Such steps may be utilized as a safety feature that leads to the disabling of the heat generating devices in instances in which the tissue temperature cannot be monitored due to a temperature sensor malfunction or defect. If the temperature sensor is not malfunctioning, the method 114 includes checking if the illumination device is malfunctioning (block 130) and, if so, the electronic devices are disabled (block 126) as before. Similarly, the method 114 includes checking for camera malfunctions (block 132) and disabling the inserted electronic devices if a malfunction is detected (block 126). However, if a malfunction of any of the electronic devices is not detected, the control circuitry enables operation of the inserted electronic devices (block 134).

It should be noted that in some embodiments, the method of FIG. 4 may include more or fewer steps than those shown in the illustrated embodiment. For example, in certain embodiments, the method may only include a check as to whether or not the temperature sensor is malfunctioning. Still further, in some embodiments, the method may include additional checks to verify the proper functioning and calibration of the inserted electronic devices. Indeed, any number and type of checks to ensure that the electronic devices are properly functioning before enabling use may be provided.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. An intubation system, comprising:
a tracheal tube configured to be placed in an airway of a patient to facilitate airflow to the patient;
a heat source coupled to the tracheal tube and configured to generate heat when the tracheal tube is disposed in the airway of the patient;
a temperature sensor coupled to the tracheal tube and configured to detect a temperature representative of a temperature of a tissue in the airway of the patient; and
a temperature control system coupled to the heat source via a lumen of the tracheal tube and configured to monitor the detected temperature and to control generation of heat from the heat source based on the detected temperature.

2. The intubation system of claim 1, wherein the temperature control system is configured to control heat generation from the heat source by modulating a duty cycle of the heat source.

3. The intubation system of claim 1, wherein the temperature control system is further configured to shut down the heat source to substantially prevent the heat source from generating additional heat when the detected temperature exceeds a desired threshold.

4. The intubation system of claim 1, wherein the heat source is at least one of a camera, an illumination device, and a transducer.

5. The intubation system of claim 1, wherein the temperature control system is further configured to detect a malfunction of the temperature sensor and to shut down the heat source when the malfunction of the temperature sensor is detected.

6. The intubation system of claim 1, wherein the temperature control system is further configured to detect a malfunction of the heat source and to shut down the heat source when the malfunction of the heat source is detected.

7. The intubation system of claim 1, wherein the temperature sensor is at least one of a thermistor, a thermocouple, and a semiconductor.

8. The intubation system of claim 1, comprising a monitor, wherein the temperature control system is configured to output a duty cycle of the heat source over time to the monitor for display to an operator.

9. A method, comprising:
intubating a patient with a tracheal tube, wherein the tracheal tube is coupled to a heat source and a temperature sensor;
detecting a temperature representative of the temperature of a tracheal tissue of the patient;
determining when the detected temperature exceeds a predefined threshold value; and
altering one or more parameters of the heat source to reduce the amount of generated heat when the detected temperature exceeds the predefined threshold.

10. The method of claim 9, comprising determining when the detected temperature exceeds a desired value and shutting down operation of the heat source when the detected temperature exceeds the desired value.

11. The method of claim 9, comprising detecting a malfunction of at least one of the temperature sensor and the heat source and shutting down operation of the heat source when a malfunction is detected.

12. The method of claim 9, comprising displaying the values of the one or more altered parameters over time to an operator on a monitor.

13. The method of claim 9, wherein the heat source is at least one of an illumination device, an imaging device, and a transducer.

14. The method of claim 9, wherein altering one or more parameters of the heat source comprising changing a duty cycle of the heat source.

15. A temperature control system for a tracheal tube, comprising:
electronic circuitry coupled to an electronic heat source configured to couple to a tracheal tube during intubation of a patient, wherein the electronic circuitry is configured to power the electronic heat source during intubation of the patient;
a temperature sensor configured to detect a local temperature level representative of a temperature level within a patient's trachea and to be disposed in a patient's trachea in a location proximate to the electronic heat source during intubation of the patient; and
control circuitry coupled to the temperature sensor and the electronic circuitry and configured to monitor the temperature level detected by the temperature sensor and to control operation of the electronic circuitry based on the detected temperature level.

16. The temperature control system of claim 15, wherein the control circuitry is configured to control the electronic circuitry to alter a duty cycle of the electronic heat source when the detected temperature level exceeds a predetermined threshold.

17. The temperature control system of claim 15, wherein the control circuitry is configured to shut down the electronic circuitry when the detected temperature level exceeds a desired threshold.

18. The temperature control system of claim 15, wherein the electronic heat source comprises at least one of an imaging device, an illumination device, and a transducer.

19. The temperature control system of claim 15, wherein the control circuitry is configured to shut down the electronic circuitry when at least one of the electronic circuitry and the temperature sensor is malfunctioning.

20. The temperature control system of claim 15, wherein the temperature sensor comprises at least one of a thermistor, a thermocouple, and a semiconductor.
